(19) United States
(12) Patent Application Publication
(10) Pub. No.: US 2021/0257105 A1
(43) Pub. Date: Aug. 19, 2021

(54) METHODS, SYSTEMS, AND COMPUTER READABLE MEDIA FOR DEFINING RISK OF ADVERSE HEALTH OUTCOMES BASED ON NON-INTRUSIVE ENERGY USAGE MONITORING

(71) Applicant: University of Virginia Patent Foundation, Charlottesville, VA (US)

(72) Inventor: Jeffrey Wayne Keller, Charlottesville (US)

(73) Assignee: University of Virginia Patent Foundation, Charlottesville, VA (US)

(21) Appl. No.: 17/271,058

(22) PCT Filed: Aug. 30, 2019
(86) PCT No.: PCT/US19/49114

§ 371 Date: Feb. 24, 2021

Related U.S. Application Data
(60) Provisional application No. 62/725,211, filed on Aug. 30, 2018.

(51) Int. Cl. G16H 50/30; G16H 40/67; G16H 10/60; A61B 5/00; G06Q 50/06

(52) U.S. Cl. CPC G16H 50/30; G16H 40/67; G16H 10/60; A61B 2503/12; A61B 5/7246; G06Q 50/06; A61B 2505/07; A61B 5/7275

(57) ABSTRACT

Methods, systems, and computer readable media for defining risk of adverse health outcomes based on non-intrusive energy usage monitoring are disclosed. One exemplary method includes receiving residential energy usage data associated with a residential location as measured by a residential meter and applying at least one load signature correlation that has been derived using previously collected energy usage data and one or more of health outcome data, the health care resource utilization data, and the clinical outcome data to the received residential energy usage data. The method further includes using an output resulting from applying the at least one load signature correlation to identify a risk or predict a likelihood of specific health outcomes or clinical events associated with at least one target entity associated with the residential location.

### PRIORITY CLAIM

This application claims the benefit of U.S. Provisional Patent Application Ser. No. 62/725,211, filed Aug. 30, 2018, the disclosure of which is incorporated herein by reference in its entirety.

### TECHNICAL FIELD

The subject matter described herein relates to the use of electrical consumption data collected at a residential location to determine a resident's predisposition to avoidable health conditions. More particularly, the subject matter described herein relates to methods, systems, and computer readable media for defining risk of adverse health outcomes based on non-intrusive energy usage monitoring.

### BACKGROUND

At present, health care expenses constitute a significant and growing percentage of the economy of the United States. Notably, several factors including an ever-increasing aging population and the utilization of a single-payer system may compel the need to implement a structural change to the health care system. Shifting reimbursement trends increasingly require health care payers and providers to more proactively identify and mitigate risk factors that drive eventual utilization of the health care system. Implemented effectively, such proactive interventions can meaningfully reduce the demand for acute medical services and their attendant costs. In recent years, numerous advancements have been made in the remote monitoring of patients with the use of electrical sensors (e.g., motion sensors, pressure sensors, video capture sensors, sound sensors, and the like) in an attempt to observe factors that may correlate with disproportionate risk of poor outcomes resulting from existing health conditions or preconditions, daily behaviors or lifestyles. The use of these types of electrical sensors for remote monitoring entails new investment, deployment, and resources for interpretation of the resulting data stream, all of which represent significant barriers to adoption at scale by either patients or providers.

Accordingly, there exists a need for methods, systems, and computer readable media for defining risk of adverse health outcomes based on non-intrusive energy usage monitoring and utilization of existing non-clinical infrastructure and data sets.

### SUMMARY

According to one aspect, the subject matter described herein includes a method for defining risk of adverse health outcomes based on non-intrusive energy usage monitoring. One exemplary method includes receiving residential energy usage data associated with a residential location as measured by a residential meter and applying at least one load signature correlation that has been derived using previously collected energy usage data and one or more of health outcome data, the health care resource utilization data, and the clinical outcome data to the received residential energy usage data. The method further includes using an output resulting from applying the at least one load signature correlation to identify a risk or predict a likelihood of specific health outcomes or clinical events associated with at least one target entity associated with the residential location.

In one example, the method further comprises pairing the residential energy usage data with one or more of health outcome data, health care resource utilization data, or clinical outcome data of at least one individual residing in the residential location. In one example of the method, the residential energy usage data is composed of aggregate household-level load data.

In one example of the method, the residential energy usage data is decomposed into a plurality of load components, using either synthetic data models to attribute energy consumption to specific appliances or classes of appliances, or hardware-enabled direct collection at the circuit level.

In one example of the method, at least one load signature correlation is applied to inform the likelihood of health outcomes associated with chronic conditions including one or more of chronic obstructive pulmonary disease, congestive heart failure, obesity, mental/behavioral health conditions, substance abuse, chronic pain, diabetes, asthma, hypertension or their corresponding preconditions.

In one example of the method, the load signature correlation or a derivative risk identifier or score is electronically provided to a health care provider, a health maintenance organization, or an insurer for the purposes of assessing risk of adverse events, allocating health care resources, predicting outcomes, controlling costs, improving outcomes, or designing health care interventions.

In one example of the method, at least one load signature correlation is derived at least in part from an analysis of a data set created by merging historical residential load data associated with at least one individual and historical health care or clinical data associated with the same at least one individual.

In one example of the method, a retrospectively validated load signature is applied prospectively for a monitoring of populations by a health system, an insurer, or other health maintenance organization using near real-time or periodic data sets.

In one example of the method, at least one load signature correlation is categorized in accordance to a plurality of risk stratification scores.

In one example of the method, at least one load signature correlation is combined with other clinical information (e.g., blood pressure, body weight, body mass index, HbA1C, comorbid diagnoses, number or frequency of prior encounters with a health system, etc.) to derive a composite signature for defining risk, targeting interventions, or predicting outcomes.

In one example of the method, at least one load signature correlation is combined with non-clinical data elements (e.g., resident's education level, employment status, family structure, access to transportation, number and relatedness of cohabitants in the home, weather forecasting data, residential zip code, etc.) to derive a composite signature for defining risk, targeting interventions, or predicting outcomes.

In one example of the method, the residential energy usage data is disaggregated to identify and monitor specific contributions of durable medical equipment for use in the residential location to monitor patient utilization and adherence to prescribed usage.

In one example of the method, the at least one load signature correlation corresponds to either aggregate or individual component loads at the residential location and comprises data indicative of a continuity of electrical consumption, irregular patterns of electrical consumption, absence of electrical consumption, or an unexpected surge of electrical consumption.

According to one aspect, the subject matter described herein includes a system for defining risk of adverse health outcomes based on non-intrusive energy usage monitoring. One exemplary system includes a public utility entity that is configured to receive residential energy usage data associated with a residential location as measured by a residential meter. The system further includes a health determination correlation (HDC) engine configured to obtain the residential usage data from the public utility entity, pair the residential energy usage data with one or more of health outcome data, health care resource utilization data, or clinical outcome data of at least one individual residing in the residential location, define at least one load signature correlation existing between the energy usage data and the one or more of the health outcome data, the health care resource utilization data, and the clinical outcome data, and use the at least one load signature correlation to define a risk or predict a likelihood of specific health outcomes or clinical events associated with at least one target entity associated with the residential location.

The subject matter described herein can be implemented in software in combination with hardware and/or firmware. For example, the subject matter described herein can be implemented in software executed by a processor. In one exemplary implementation, the subject matter described herein can be implemented using a non-transitory computer readable medium having stored thereon computer executable instructions that when executed by a processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

Preferred embodiments of the subject matter described herein will now be explained with reference to the accompanying drawings, wherein like reference numerals represent like parts, of which:

FIG. 1 is a block diagram illustrating an exemplary system for methods, systems, and computer readable media for defining risk of adverse health outcomes based on non-intrusive energy usage monitoring according to an embodiment of the subject matter described herein; and

FIG. 2 is a flow chart illustrating an exemplary process for methods, systems, and computer readable media for defining risk of adverse health outcomes based on non-intrusive energy usage monitoring according to an embodiment of the subject matter described herein.

### DETAILED DESCRIPTION

In accordance with the subject matter disclosed herein, systems, methods, and computer readable media for defining risk for adverse health outcomes based on non-intrusive energy usage monitoring are provided. Specifically, the disclosed subject matter presents a system for implementing non-intrusive appliance load monitoring that positively identifies an intersection existing between residential energy usage and a resident's predisposition to avoidable health conditions or encounters. As such, the disclosed system affords an opportunity to identify at-risk individuals or sub-populations based on residential energy usage patterns. Utilizing data generated from such a system enables a health care provider, case manager, social worker, or other health care professional to address and provide appropriate clinical intervention at an early stage, thereby avoiding costly health encounters and adverse health outcomes. For example, the disclosed subject matter may utilize data indicative of the frequency and/or duration in which an appliance or device is operated within a residential location to identify at-risk residents. For example, if a television is powered on for a significant percentage of the day, or until an extremely early hour, the target entity (e.g., resident) residing at that residential location may be designated as having a greater risk of experiencing adverse health events. Exemplary systems and methods are described below.

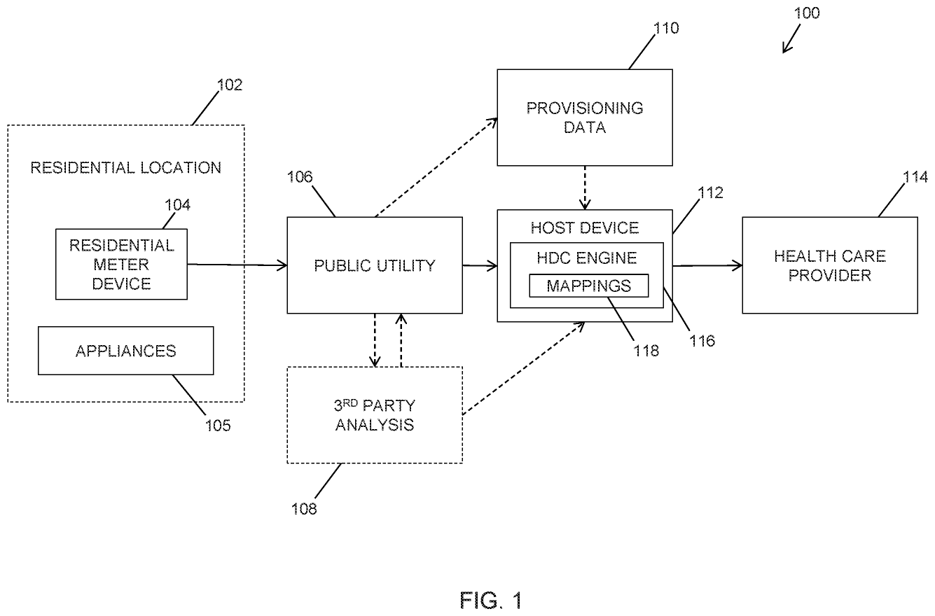

FIG. 1 depicts a system 100 that includes a residential location 102, a public utility entity 106, a host device 112, and a health care provider entity 114. In some embodiments, residential location 102 can comprise any type of residence, such as a single-family home, an apartment unit, or the like. Residential location 102 can further include a plurality of load devices or appliances 103 that are plugged into the home electrical wiring system of residential location 102. Residential location 102 includes a residential meter device 104, such as a smart meter device, that is responsible for monitoring and/or collecting the residential energy usage data associated with the residential location. Notably, residential meter device 104 and public utility entity 106 comprise an AMI that can be configured to monitor and collect energy usage data associated with any number of serviced residential locations. In some embodiments, residential meter device 104 may be communicatively connected to a public utility entity 106 using local area networks and/or public cellular networks, such that usage data can be readily communicated between public utility entity 106 and the sites of usage (e.g., residential location 102 and residential meter device 104). Similarly, radio frequency (RF) based AMI networks or wide area AMI networks can also be used to facilitate the communicative connection between the residential meter device 104 and public utility entity 106. In particular, residential meter device 104 can be configured to provide aggregated residential energy usage data that is representative of the electrical consumption associated with load devices and appliances 105 to public utility entity 106 for further processing (e.g., disaggregation), analysis, or distribution. In some embodiments, public utility entity 106 can be adapted to ‘ping’ meter devices on a periodic basis in order to ascertain a point in time energy usage. For example, public utility entity 106 may send a request message to residential meter device 104 at a frequency of every 5 minutes or every 60 minutes. Notably, this frequency can be predefined to any time period value.

In some embodiments, after receiving the energy usage data communicated by residential meter device 104, public utility entity 106 may be configured to disaggregate the energy usage data into electrical load components. In some alternate embodiments, public utility entity 106 can forward the aggregated residential energy usage data to a third-party analysis entity 108.

Regardless if disaggregation of the energy usage data is conducted locally or by third-party analysis entity 108, public utility entity 106 is ultimately configured to provide the disaggregated residential energy usage data to host device 112. In some embodiments, public utility entity 106 and third-party analysis entity 108 can each be provisioned with a disaggregation engine that is configured to utilize analytics that permit the synthetic disaggregation of household loads into components (e.g., individual identified appliances or category type of appliances, such as cooking appliances, refrigeration appliances, washing/drying appliances, lighting appliances, audiovisual entertainment devices, and the like). Specifically, the disaggregation engine can utilize synthetic data models to decompose residential energy usage data into a plurality of load components to attribute energy consumption to specific appliances or classes of appliances. Alternatively, the disaggregation of household loads into components can entail hardware-enabled direct collection at the circuit level.

In some embodiments, public utility entity 106 can be configured to utilize the energy usage data in its aggregated form (prior to or sans disaggregation). For example, aggregated residential energy usage data comprising a particular amount of energy (or load pattern) used during specific times of the day can be determined and monitored (e.g., 1.5 kilowatts consistently used between 5 pm to 10 pm). Further, the disclosed subject matter may also ascertain whether the amount of energy consumed during a particular time period of the day is consistently the same for a number of days of the week or month. Notably, irregular energy usage consumed during a particular time of a day is consistent with a possible adverse health condition. Likewise, regular patterns of energy usage associated with a residential location or household that is collectively characterized as a low health risk can be used to establish an aggregated energy load signature pattern.

Host device 112 may include a special purpose machine that is configured to determine a resident's risk of specific health outcomes based on non-intrusive energy usage monitoring. In some embodiments, host device 112 includes one or more processors and memory that are collectively utilized to support a health determination correlation (HDC) engine 116 (described below). In some embodiments, the processor(s) may include a central processing unit (e.g., a single core or multiple processing cores), a microprocessor, a microcontroller, a network processor, an application-specific integrated circuit (ASIC), or the like. Likewise, the memory may comprise random access memory (RAM), flash memory, a magnetic disk storage drive, and the like. In some embodiments, the memory may be configured to store HDC engine 116. In some embodiments, the processor(s) and memory can be managed by a hypervisor and serve as the underlying hardware for supporting virtual machines that host HDC engine 116.

FIG. 1 further depicts host device 112 as receiving provisioning data 110. In some embodiments, provisioning data 110 includes historical energy usage information associated with a plurality of target entities (e.g., persons who are both a resident and patient). In some instances, the historical energy usage information can be provided by public utility entity 106. For example, the historical energy usage data (e.g., historical AMI data) can include 18 months of electrical consumption information corresponding to a plurality of residential locations corresponding to a respective number of target entities. Provisioning data 110 may also include historical health record or utilization information (e.g., health insurance claims) associated with the same target entities. For example, provisioning data 110 can include emergency department records (e.g., emergent encounters) and inpatient admission data pertaining to the same target entities during the same/retrospective time frame (e.g., 18 months) as the historical energy usage data. In some embodiments, the coupling of the health care and utility data is important for deriving one or more correlations between the load signatures and health risks. After the correlations have been derived and identified, energy usage data can be scanned for the presence (or absence) of load signatures. Notably, historical health record data and historical energy usage information do not need to be reincorporated or continuously collected after the appropriate correlation(s) is derived/established (as described below). For example, after a load signature correlation is established using the historical energy usage data and the historical health record data, the derived load signature correlation can be applied to other areas and other target entities where health record data (i.e., health outcome data, health care resource utilization data, and/or clinical outcome data) may not be available or obtainable. As such, the disclosed subject matter can apply the previously derived load signature correlation to energy usage data in such a scenario in order to identify a load signature that is indicative of a potential health risk.

In some embodiments, provisioning data 110 can be utilized by host device 112 to generate a plurality of load signature correlations (e.g., depicted as mappings 118 in FIG. 1). More specifically, HDC engine 116 can utilize provisioning data 110 to derive at least one load signature correlation that exists between the residential energy usage data and one or more of health outcome data, health care resource utilization data, and clinical outcome data pertaining to one or more target entities. As used herein, health outcome data refers to health condition information pertaining to a target entity (e.g., diabetes, hypertension, asthma, etc.) and health care resource utilization data refers to data pertaining to the actual resources that are utilized as a result of the target entity's heath outcome data (e.g., ambulance ride, emergency room visit, and/or hospitalization). Similarly, clinical outcome data refers to the underlying clinical condition (e.g., diabetic ketoacidosis, myocardial infarction), the treatment of which results in consumption of health care resources.

HDC engine 116 can subsequently use a load signature correlation to serve as mechanism that produces defined risk or predicted likelihood of a specific health outcome for a clinical event for an associated individual based on residential energy usage data that is received as input (e.g., in aggregated or disaggregated form). HDC engine 116 can also be configured to employ machine learning mechanisms to subsequently utilize this input and output data to further derive existing load signature correlations or establish new load signature correlations.

In some embodiments, machine learning algorithms can be utilized to build a mathematical model that is based on a bi-dimensional data set comprising the historical energy usage data and the historical health record or utilization information corresponding to a common set of individuals (or population) over a common time period (e.g., 12-24 months). Notably, the two historical datasets described above can be exposed to machine learning approaches that are configured to determine a load signature correlation existing between residential energy usage and health care utilization.

In some embodiments, the HDC engine comprises a machine learning artificial intelligence (AI) algorithm that uses a multi-layer N-stage deep learning or other neural network, classification tree, or other machine learning approach to formulate a non-linear prediction model that can process and cross-validate the historical energy usage data and the historical health record or utilization information. For example, a machine learning based HDC engine can train a neural network that performs the various functions described herein while utilizing highly parallel computers or processors, such as graphical processing units (GPUs).

In some embodiments, patterns of energy consumption for a single household can be analyzed by HDC engine 116 over a period of time for consistency. Notably, HDC engine 116 can be configured to consider external factors (e.g., seasons, weather, geographical location, and the like) in conjunction with the stability of the energy consumption pattern representing a factor to be correlated with health outcomes, likelihood of utilization of health care resources, or clinical outcomes. Likewise, HDC engine 116 can be adapted to analyze patterns of energy consumption for a single household against similar patterns associated with the overall grid or a subset of the overall grid (e.g., total residential consumption versus consumption by industrial and/or commercial customers within the overall grid) for consistency with or deviation from a larger pattern. Further, HDC engine 116 can consider customized discrete groups/subsets, such as residential locations that consume over (or under) a particular threshold level of energy usage.

In some embodiments, HDC engine 116 utilizes the derived load signature correlations as a processing mechanism to determine a health score or risk identifier based on residential energy usage data that HDC engine 116 received as input. For example, HDC engine 116 can receive electrical load component data characterized by various electrical metrics, such as electrical transient data, impulse data, and/or steady-state data. Upon receiving the residential energy usage data (in either aggregated or disaggregated form), HDC engine 116 may identify the particular electrical device or appliance (or category type of appliances/devices) that is being utilized in the residential location as well as the time of day and duration that the appliance/device is being operated. After discerning this device activity information, HDC engine 116 is configured to utilize one or more load signature correlations to determine a resident's corresponding health assessment data. Examples of health assessment data that can be determined include i) equating the continuity of load element usage as being indicative of excessively sedentary lifestyle, ii) equating significant changes in load patterns as being indicative of possible depressive or manic states, iii) equating irregular load patterns as being indicative of possible apnea or insomnia, iv) equating non-usage of cooking appliances as being indicative of possible unhealthy eating, v) equating long periods of unchanged load patterns as being indicative of possible substance abuse, and the like. Table 1 below depicts examples of different health assessment data categories.

TABLE 1

| | |
|---|---|
| Congestive Heart | Continuity of load elements indicative of |
| failure | excessively sedentary lifestyle; lack of |
| | hourly changes corresponding with |
| | cooking, eating, travel. |
| Behavioral Health | Significant changes in load patterns |
| | corresponding to depressive |
| | or manic states. |
| Chronic Obstructive | Irregular load patterns such as television |
| Pulmonary Disease | during hours when sleep is expected |
| (COPD) | indicating apnea or insomnia |
| Diabetes | Non-usage of cooking appliances that |
| | may indicate the individual is |
| | relying on prepared or processed foods |
| | that may not provide appropriately |
| | balanced nutrition. Continuity of |
| | load elements indicative of |
| | excessively sedentary lifestyle |
| Hypertension | Continuity of load elements indicative |
| | of excessevely sedentary lifestyle |
| Obesity | Continuity of load elements indicative |
| | of excessively sedentary lifestyle |
| Asthma | Unexpected reduction in HVAC |
| | usage seasonally that could indicate |
| | household financial shortfall |
| | and inability to pay utility bills |
| Coronary Artery | Continuity of load elements indicative |
| Disease | of excessively sedentary lifestyle |
| Substance Abuse | Long periods of unchanged load pattern; |
| | load pattern decoupled from normal |
| | 24 hr schedule |

In some embodiments, HDC engine 116 is configured to utilize at least one load signature that is used to identify individuals at differential risk of adverse health outcomes or avoidable encounters with the health system from among a larger population (e.g., a population comprising insured lives). Further, the HDC engine 116 may be adapted to identify individuals in such a manner either using a periodic or continuous re-analysis based on continuously collected residential energy usage data.

Moreover, HDC engine 116 can also be configured in some embodiments to assess the presence of at least one load signature in the monitoring of specific patients that identified to be at high risk in real-time for status (e.g., post-discharge from a hospital for fall risk).

In some embodiments, HDC engine 116 is further configured to generate a score or risk identifier corresponding to the determined health assessment data generated from the load signature correlations. For example, HDC engine 116 is configured to determine a score or risk identifier pertaining to possible exacerbation of known clinical conditions, or the advancement of pre-conditions leading to a clinically manifest state of conditions such as congestive heart failure, hypertension, and/or obesity based on the degree of correlation (e.g., measured amount and duration of energy usage data associated with a particular load element, such as a television or personal computer). In some embodiments, at least one load signature correlation is used to guide interventions by health care providers, insurers, or health maintenance organizations to preempt costly and avoidable encounters with the health system through proactive measures. Exemplary proactive measures that can be taken include calling the target entity, request that target entity provide biometric measurements, conducting a home visit, request that target entity adjust the dosage of a prescribed mediation (or inquire as to the effectiveness of the medication), ensure prescription for target entity is filled at a pharmacy, visits from an off-duty paramedic, schedule a clinic or office visit, and the like.

Once the health indication data is generated by HDC engine 116, host device 112 is configured to send the score or risk identifier output to one or more recipients. For example, host device 112 can transmit or forward an electronic message or report (e.g., electronic mail, SMS, MMS, etc.) to health care provider entity 114. Health care provider entity 114 as depicted in FIG. 1 may include a health care system entity, an insurer, or some other health maintenance organization or entity. Notably, health care provider entity 114 can utilize the score or risk identifier to target interventions or predict health outcomes for the target entity/patient/resident. As such, health care provider entity 114 can utilize the output of HDC engine 116 to better address and/or pre-empt a major clinical event. In some embodiments, HDC engine 116 is configured to utilize any suitable range or schema of scores (e.g., ‘0’-‘100’ or Low, Medium, and High risk) that is representative of the degree of health risk.

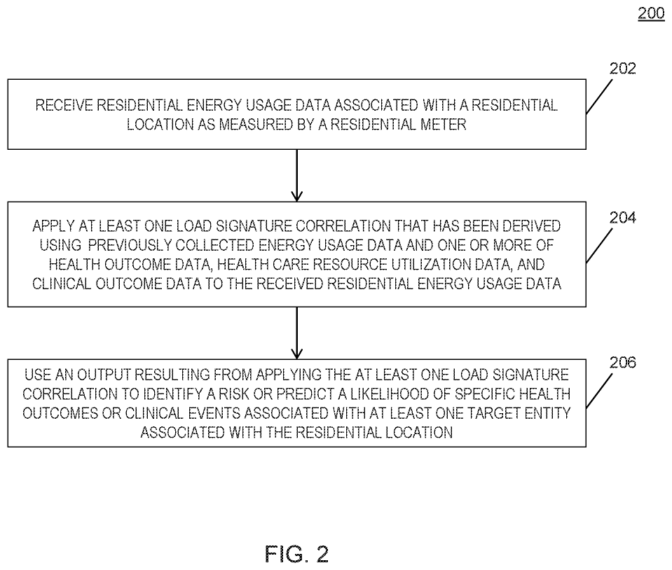

FIG. 2 is a flow chart illustrating an exemplary method 200 defining risk of adverse health outcomes based on non-intrusive energy usage monitoring according to an embodiment of the subject matter described herein. In some embodiments, blocks 202-206 of method 200 may represent an algorithm or process performed by a health determination correlation (HDC) engine that is stored in memory and executed by one or more processors of a host device.

In block 202, residential energy usage data associated with a residential location as measured by a residential meter device is received. In some embodiments, the energy usage data corresponding to the residential location is measured by a smart meter that is configured to collectively record the electrical power consumed by devices and appliances located at the residential location. The smart meter may then forward the collected aggregation of the residential energy usage data over a period of time (e.g., on a periodic basis) to the public utility responsible for servicing the residential location. In some embodiments, the public utility entity and the plurality of smart meter devices deployed at the serviced residential locations make up an Advanced Metering Infrastructure. Prior to receipt by a host device and/or HDC engine, the residential energy usage data can be disaggregated into separate component loads corresponding to the separate appliances and devices located in the residential location. Notably, the aggregated residential energy usage data may be disaggregated by either the public utility or a third-party analysis entity prior to the host device and/or HDC engine receiving the residential energy usage data. In some embodiments, the residential energy usage data remains in its aggregated form when processed by the public utility entity (e.g., the residential energy usage data is not actually disaggregated at any time).

In block 204, at least one load signature correlation that has been derived using previously collected energy usage data and one or more of health outcome data, the health care resource utilization data, and the clinical outcome data is applied to the received residential energy usage data. In some embodiments, the load signature correlation is applied by the HDC engine to the energy usage data collected and provided by the residential meter device. As indicated above, the load signature correlation has been derived using historical energy usage data and historical health care data. More specifically, the at least one load signature can be derived by pairing the residential energy usage data and health care data. Specifically, the residential energy usage data can be paired with one or more health outcome data, health care resource utilization data, or clinical outcomes data of at least one individual residing in the residential location. In some embodiments, the HDC engine may be initially pre-loaded and/or provisioned with health care outcome data corresponding to a plurality of target entities (e.g., a resident/patient) as well as residential energy usage data corresponding to those same target entities. In some embodiments, the residential energy usage data and the one or more health outcome data, health care resource utilization data (e.g., emergent encounters, inpatient admissions, etc.), or clinical outcomes data can be paired or linked using both a common time frame (e.g., last 18 months) and a common target entity (i.e., a common resident/patient associated with all sets of the paired data).

In other examples, the load signature correlation may be derived at least in part from a mapping of a data set created by merging historical residential location data associated with at least one individual (e.g., target entity) and historical health care or clinical data associated with the same at least one individual. In other embodiments, a load signature correlation can be retrospectively validated and subsequently applied prospectively for the monitoring of populations by a health system, insurer, or other health maintenance organization using near time or periodic data sets. Likewise, a load signature correlation can be retrospectively validated and subsequently applied prospectively for the monitoring of specific individuals known to a health system, an insurer, or other health maintenance organization using near real-time or periodic data sets. In some embodiments, the HDC engine is configured to conduct the retrospective validation and prospective application of the load signature correlation.

In some embodiment, the initial loading of these two categories of data indicated in block 204 enables the HDC engine to utilize machine learning capabilities to construct a control set model that comprises at least one load signature correlation. In particular, the load signature correlation(s) may constitute mappings or correlations that define a nexus between disaggregated residential energy data (e.g., one or more component device loads associated with a respective particular device/appliance in the residential location) and the health outcome data, health care resource utilization data, or clinical outcomes data corresponding to a resident of a residential location. Once the control set model is constructed, the model can be utilized by the HDC engine to determine or predict a current target entity's (e.g., resident/patient) health care outcome based on energy usage data input received from the target entity's residential location. Notably, the use of historical data is no longer required once the load correlation(s) is established by the HDC engine.

In some embodiments, at least one load signature correlation can correspond to an individual component load (or appliance type or category) at a residential location and comprises data that can be indicative of a continuity of electrical consumption, irregular patterns of electrical consumption, absence of electrical consumption, or an unexpected surge of electrical consumption.

In block 206, an output resulting from applying the at least one load signature correlation is used to identify (or define) a risk or predict a likelihood of specific health outcomes or clinical events associated with at least one target entity (e.g., individual and/or resident) associated with the residential location. For example, at least one load signature correlation can be applied by the HDC engine to inform the likelihood of health outcomes associated with chronic conditions such as chronic obstructive pulmonary disease, congestive heart failure, obesity, mental/behavioral health conditions, substance abuse, chronic pain, diabetes, asthma, hypertension, or their respective corresponding preconditions. In some embodiments, a derivative health risk identifier or score generated by the HDC engine using the load signature correlation(s) is electronically provided to a health care provider, a health maintenance organization, or an insurer. For example, the host device and/or HDC engine can be configured to send an electronic message to a health care provider in the event a particular health risk identifier or score exceeds a predefined threshold level or designation. Notably, the electronic message can be provided for the purposes of assessing risk of adverse events, allocating health care resources, predicting outcomes, controlling costs, improving outcomes, or designing health care interventions as related to the resident/patient. In some instances, the health and education data that is provided to the health care professional or patient advocate that is responsible for monitoring the individual patient can be acted upon by the entities. Further, the health care professional may be financially compelled to provide the service since the health care provider bears the responsibility for outcomes under risk-based contracts.

In other embodiments, at least one load signature correlation may be combined with other clinical information to derive a composite signature correlation for defining risk, targeting interventions, or predicting outcomes. Similarly, at least one load signature correlation can also be combined with non-clinical data elements in order to derive a composite signature correlation for defining risk, targeting interventions, or predicting outcomes. For example, data related to a resident's education level, employment status, family structure, access to transportation, number and relatedness of cohabitants in the home, weather forecasting, residential zip code data, and the like can be combined with at least one load signature correlation to derive a composite signature correlation that can be used by the HDC engine to determine a resident's health outcomes.

It will be understood that various details of the subject matter described herein may be changed without departing from the scope of the subject matter described herein. Furthermore, the foregoing description is for the purpose of illustration only, and not for the purpose of limitation.

What is claimed is:

1. A method for defining risk of adverse health outcomes based on non-intrusive energy usage monitoring comprising:
receiving residential energy usage data associated with a residential location as measured by a residential meter;
applying at least one load signature correlation that has been derived using previously collected energy usage data and one or more of health outcome data, health care resource utilization data, and clinical outcome data to the received residential energy usage data; and
using an output resulting from applying the at least one load signature correlation to identify a risk or predict a likelihood of specific health outcomes or clinical events associated with at least one target entity associated with the residential location.

2. The method of claim 1 further comprising pairing the residential energy usage data with one or more of health outcome data, health care resource utilization data, or clinical outcome data of at least one individual residing in the residential location.

3. The method of claim 1 wherein the residential energy usage data is composed of aggregate household-level load data.

4. The method of claim 1 wherein the residential energy usage data is decomposed into a plurality of load components using either synthetic data models to attribute energy consumption to specific appliances or classes of appliances, or hardware-enabled direct collection at a circuit level.

5. The method of claim 1 wherein at least one load signature correlation is applied to inform a likelihood of health outcomes associated with chronic conditions including one or more of chronic obstructive pulmonary disease, congestive heart failure, obesity, mental/behavioral health conditions, substance abuse, chronic pain, diabetes, asthma, hypertension or their corresponding preconditions.

6. The method of claim 1 wherein the load signature correlation or a derivative risk identifier or score is electronically provided to a health care provider, a health maintenance organization, or an insurer for the purposes of assessing risk of adverse events, allocating health care resources, predicting outcomes, controlling costs, improving outcomes, or designing health care interventions.

7. The method of claim 1 wherein load signature correlation is derived at least in part from an analysis of a data set created by merging historical residential load data associated with at least one individual and historical health care or clinical data associated with the same at least one individual.

8. The method of claim 1 wherein a retrospectively validated load signature is applied prospectively for a monitoring of populations by a health system, an insurer, or other health maintenance organization using near real-time or periodic data sets.

9. The method of claim 1 wherein a retrospectively validated load signature is applied prospectively for a monitoring of specific individuals known to a health system, an insurer, or other health maintenance organization using near real-time or periodic data sets.

10. The method of claim 1 wherein at least one load signature correlation is categorized in accordance to a plurality of risk stratification scores.

11. The method of claim 1 wherein at least one load signature correlation is combined with other clinical information to derive a composite signature for defining risk, targeting interventions, or predicting outcomes.

12. The method of claim 1 wherein at least one load signature correlation is combined with non-clinical data elements to derive a composite signature for defining risk, targeting interventions, or predicting outcomes.

13. The method of claim 1 wherein the residential energy usage data is disaggregated to identify and monitor specific contributions of durable medical equipment for use in the residential location to monitor patient utilization and adherence to prescribed usage.

14. The method of claim 1 wherein at least one load signature correlation corresponds to either aggregate or individual component loads at the residential location and comprises data indicative of a continuity of electrical consumption, irregular patterns of electrical consumption, absence of electrical consumption, or an unexpected surge of electrical consumption.

15. A system for defining risk of adverse health outcomes based on non-intrusive energy usage monitoring comprising:
a public utility entity that is configured to receive residential energy usage data associated with a residential location as measured by a residential meter; and
a health determination correlation (HDC) engine configured to obtain the residential energy usage data from the public utility entity, apply at least one load signature correlation that has been derived using previously collected energy usage data and one or more of health outcome data, health care resource utilization data, and clinical outcome data, and use an output resulting from applying the at least one load signature correlation to define a risk or predict a likelihood of specific health outcomes or clinical events associated with at least one target entity associated with the residential location.

16. The system of claim 15 wherein the HDC engine is configured to pair the residential energy usage data with one or more of health outcome data, health care resource utilization data, or clinical outcome data of at least one individual residing in the residential location.

17. The system of claim 15 wherein the residential energy usage data is composed of aggregate household-level load data.

18. The system of claim 15 wherein load signature correlation is derived at least in part from an analysis of a data set created by merging historical residential load data associated with at least one individual and historical health care or clinical data associated with the same at least one individual.

19. The system of claim 15 wherein a retrospectively validated load signature is applied prospectively for a monitoring of specific individuals known to a health system, an insurer, or other health maintenance organization using near real-time or periodic data sets.

20. A non-transitory computer readable medium having stored thereon executable instructions that when executed by a processor of a computer control the computer to perform steps comprising:
receiving residential energy usage data associated with a residential location as measured by a residential meter;
applying at least one load signature correlation that has been derived using previously collected energy usage data and one or more of health outcome data, health care resource utilization data, and clinical outcome data to the received residential energy usage data; and
using an output resulting from applying the at least one load signature correlation to identify a risk or predict a likelihood of specific health outcomes or clinical events associated with at least one target entity associated with the residential location.
